(19) United States
(12) Patent Application Publication
(10) Pub. No.: US 2011/0004083 A1
(43) Pub. Date: Jan. 6, 2011

(54) Method and Device for Measuring Blood Flow Velocity

(76) Inventors: Hsuehkuan Lu, Taiwan (CN); Chihyi Lu, Taiwan (CN)

Correspondence Address: Dr. BANGER SHIA;Patent Office of Bang Shia, 102 Lindencrest Ct., Sugar Land, TX, 77479-5201, US

(21) Appl. No.: 12/597,226

(22) PCT Filed: Apr. 24, 2008
(86) PCT No.: PCT/CN08/00839

§ 371 Date: Jul. 27, 2010

(30) Foreign Application Priority Data
Apr. 24, 2007 (CN) 200710101229.4

(51) Int. Cl. A61B 5/0265; A61B 5/1455; A61B 6/00

(52) U.S. Cl. 600/328; 600/504; 600/323; 600/479

(57) ABSTRACT

A method for measuring blood flow velocity comprises the steps of: placing a first and second blood vessel signal detectors on a body of a person to be measured in such a manner that the first and second blood vessel signal detectors are located a predetermined distance from each other; using the first and second blood vessel signal analyzers to record the blood signal at a predetermined time interval; setting an interval of time from the moment a specific blood vessel signal appears in a record of the first blood vessel signal analyzer to the moment the specific blood vessel signal appears in a record of the second blood vessel signal analyzer to be a predetermined time period; and dividing a value of the predetermined distance by a value of the predetermined time period can get a blood flow velocity.

### BACKGROUND OF THE INVENTION

1. Field of the Invention

The present invention relates to a method and device for measuring blood flow velocity, and more particularly to a method and device for measuring blood flow velocity of a person to be measured.

2. Description of the Prior Art

Blood circulation system is a main pipeline used to transport nutrients and metabolic waste, hence, blood flow velocity is closely related to human being's health. If blood flow velocity is too slow, metabolism will be deteriorated, and the metabolic product produced cannot be discharged quickly and will be accumulated in the human body, which is likely to cause fatigue and even illness. Therefore, blood flow velocity measurement is an important indicator for diagnosis.

There are many conventional methods of measuring blood flow velocity, for example, one method is to inject photosensitizing agents into the blood vessel in question, and then figure out the blood flow velocity indirectly by photography using camera or video. Another method is the use of Doppler blood flowmeter which works on the Doppler effect: the wavelength of radiation shifts because of the relative motion of the light source and the observer, the waves before the moving wave source are compressed, their wavelength is therefore relatively short and wave frequency is relatively high, and contrarily, the waves after the moving wave source have a relatively long wavelength and low frequency. Today, ultrasonic and infrared have been used together with Optical Doppler tomography (ODT) to measure the velocity of blood flow. Since the blood flow in blood vessel in question can cause Doppler frequency shift of the back-scattered photons, and the amount of shift in frequency is in proportion to the velocity of the blood. In this way, the blood flow velocity can be calculated. The Doppler frequency shift is accurate for measuring the motion of a specific object though, such technique is not easy to implement because the blood flow is complicated by the fact that it is pulsatile. Furthermore, the Doppler blood flowmeter is too expensive to be widely used.

The present invention has arisen to mitigate and/or obviate the afore-described disadvantages.

### SUMMARY OF THE INVENTION

The primary object of the present invention is to provide a method and device for quickly measuring blood flow velocity of a person to be measured.

To achieve the above object, the method for measuring blood flow velocity in accordance with the present invention comprises the steps of: placing a first and second blood vessel signal detectors on a body of a person to be measured, wherein the first blood vessel signal detector is electrically connected to a first blood vessel signal analyzer, the second blood vessel signal detector is electrically connected to a second blood vessel signal analyzer, and the first and second blood vessel signal detectors are located a predetermined distance from each other; using the first and second blood vessel signal analyzers to record the blood signal of the person to be measured at a predetermined time interval; setting an interval of time from the moment a specific blood vessel signal appears in a record of the first blood vessel signal analyzer to the moment the specific blood vessel signal appears in a record of the second blood vessel signal analyzer to be a predetermined time period; and dividing a value of the predetermined distance by a value of the predetermined time period can get a blood flow velocity between the first and second blood vessel signal detectors.

The blood flow velocity obtained is an average blood flow velocity between the first and second blood vessel signal detectors.

The first and second blood vessel signal detectors are connected by a connecting member.

The connecting member is a length-adjustable link rod or telescopic rod.

The first and second blood vessel signal detectors are of invasive or non-invasive type.

The first and second blood vessel signal detectors are of clamp or loop type.

The first and second blood vessel signal analyzers are used to analyze signals produced by blood flow and selected from the group consisting of oxy-meter, oxyhemoglobin analyzer, deoxyhemoglobin analyzer, carboxyhemoglobin analyzer, methemoglobin analyzer, and carbon dioxide meter.

The first and second blood vessel signal analyzers are used to analyze wave signals of blood.

A light beam of a predetermined wavelength is projected into blood vessel of the person to be measured to enable a specific type of blood cells or blood ingredient to produce an optical rotation signal which is used as a blood vessel signal value of the person to be measured and received by the blood vessel signal analyzers.

To achieve the above object, a device for measuring blood flow velocity in accordance with the present invention comprises: a controller, a first blood vessel signal analyzer, a second blood vessel signal analyzer, a first and second blood vessel signal detectors, wherein a first blood signal detected and analyzed by the first blood vessel signal detector and the first blood vessel signal analyzer and a second blood signal detected and analyzed by the second blood vessel signal detector and the first blood vessel signal analyzer are all recorded by the controller, and then the controller calculate the blood flow velocity of the person to be measured.

The blood flow velocity obtained is an average blood flow velocity between the first and second blood vessel signal detectors.

The first and second blood vessel signal detectors are connected by a connecting member.

The connecting member is a length-adjustable link rod or telescopic rod.

The first and second blood vessel signal detectors are of invasive or non-invasive type.

The first and second blood vessel signal detectors are of clamp or loop type.

The first and second blood vessel signal analyzers are used to analyze signals produced by blood flow and selected from the group consisting of oxy-meter, oxyhemoglobin analyzer, deoxyhemoglobin analyzer, carboxyhemoglobin analyzer, methemoglobin analyzer, and carbon dioxide meter.

The first and second blood vessel signal analyzers are used to analyze wave signals of blood.

A light beam of a predetermined wavelength is projected into blood vessel of the person to be measured to enable a specific type of blood cells or blood ingredient to produce an optical rotation signal which is used as a blood vessel signal value of the person to be measured and received by the blood vessel signal analyzers.

To achieve the above object, the method for measuring blood flow velocity in accordance with the present invention comprises the steps of: placing a first and second blood vessel signal detectors on a body of a person to be measured, wherein the first blood vessel signal detector is electrically connected to a first blood vessel signal analyzer, the second blood vessel signal detector is electrically connected to a second blood vessel signal analyzer, and the first and second blood vessel signal detectors are located a predetermined distance from each other; using the first and second blood vessel signal analyzers to record the blood oxygen saturation of the person to be measured at a predetermined time interval; setting an interval of time from the moment a specific signal appears in a record of the first blood vessel signal analyzer to the moment the specific blood vessel signal appears in a record of the second blood vessel signal analyzer to be a predetermined time period; and dividing a value of the predetermined distance by a value of the predetermined time period can get a blood flow velocity between the first and second blood vessel signal detectors.

To achieve the above object, a device for measuring blood flow velocity in accordance with the present invention comprises: a controller, a first blood vessel signal analyzer, a second blood vessel signal analyzer, a first and second blood vessel signal detectors, wherein a first blood signal detected and analyzed by the first blood vessel signal detector and the first blood vessel signal analyzer and a second blood signal detected and analyzed by the second blood vessel signal detector and the first blood vessel signal analyzer are all recorded by the controller, and then the controller calculate the blood flow velocity of the person to be measured.

The first and second blood vessel signal detectors are connected by a connecting member, and the connecting member is a length-adjustable link rod or telescopic rod.

The first and second blood vessel signal detectors are not limited to invasive or non-invasive type.

The first and second blood vessel signal analyzers are used to analyze signals produced by blood flow and selected from the group consisting of oxy-meter, oxyhemoglobin analyzer, deoxyhemoglobin analyzer, carboxyhemoglobin analyzer, methemoglobin analyzer, carbon dioxide meter, other detectors or analyzers for detecting or analyzing substances level or signal in blood, or devices for analyzing the blood wave signal.

Based on the abovementioned technology, the method for measuring the blood flow velocity in accordance with the present invention is such that the person to be measured wears two blood vessel signal detectors which are located a predetermined distance from each other and placed upstream and downstream of the predetermined position on the user's body where the blood flow velocity is to be measured, along the blood flow direction. Since the blood signal will be transmitted in the blood flow direction, in this way, a predetermined time period can be obtained, and the blood flow velocity at the predetermined position on the user's body can be figured out. This method can figure out the blood flow velocity at the predetermined position on the user's body very quickly without requiring the user to do any movement or to respond to external stimuli. Hence, it is cheap but accurate and has the potential for wide application.

### BRIEF DESCRIPTION OF THE DRAWINGS

FIG. 1 is a graph showing the blood oxygen saturation values recorded by the method and device for measuring blood flow velocity in accordance with the present invention during a plurality of time intervals, when blood flow velocity is measured;

FIG. 2 is an illustrative view of a device for measuring blood flow velocity in accordance with a first embodiment of the present invention;

FIG. 3 is an illustrative view of a device for measuring blood flow velocity in accordance with a second embodiment of the present invention; and

FIG. 4 is an illustrative view of a device for measuring blood flow velocity in accordance with a third embodiment of the present invention.

### DETAILED DESCRIPTION OF THE PREFERRED EMBODIMENTS

The present invention will be clearer from the following description when viewed together with the accompanying drawings, which show, for purpose of illustrations only, the preferred embodiment in accordance with the present invention.

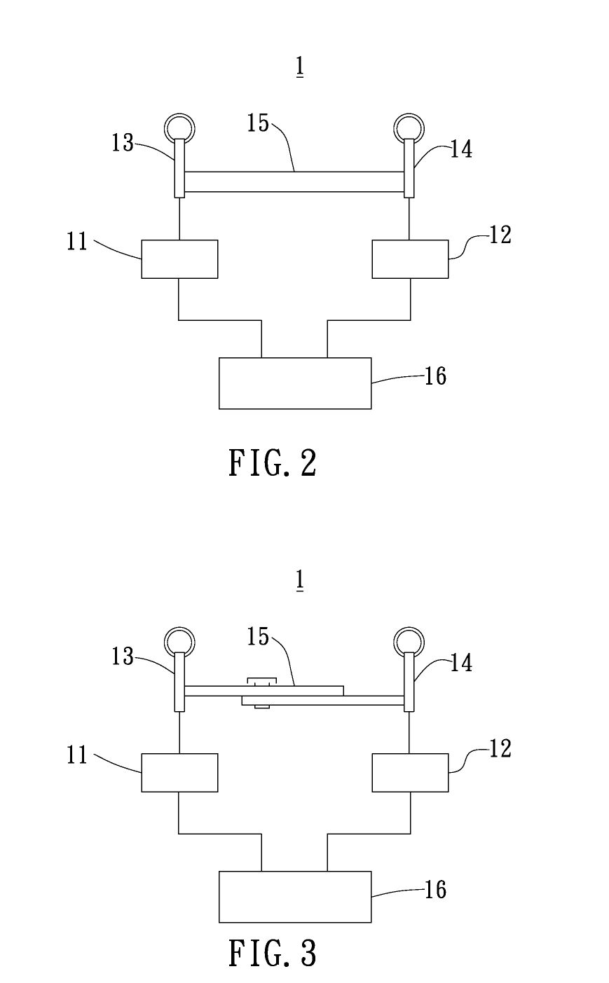

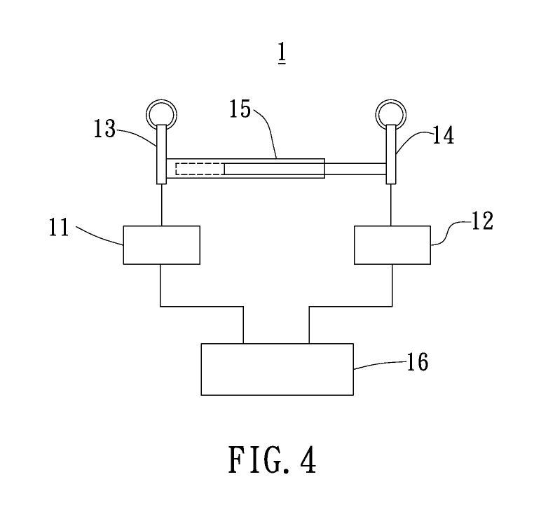

Referring to FIG. 2, a method for measuring blood flow velocity in accordance with the present invention is illustrated and comprises: placing a first and second blood vessel signal detectors 13 and 14 on the person to be measured (the detectors are to be worn by the person to be measured at the predetermined position at which the blood flow velocity is to be measured, preferably, the detectors are arranged upstream and downstream of the predetermined position, respectively, along the blood flow direction. The first blood vessel signal detector 13 is electrically connected to a first blood vessel signal analyzer 11, and the second blood vessel signal detector 14 is electrically connected to a second blood vessel signal analyzer 12. The first and second blood vessel signal analyzers 11, 12 are used to detect the signal produced by the blood flow in the human body's blood vessels (including heat vessel and body vessel) by projecting a light beam of a predetermined wavelength (such as infrared) into the vessel of a person to be measured in an invasive or non-invasive manner to enable specific type of blood cell or blood ingredient in the blood vessels of the person A to be measured to produce a special optical rotation signal. Since different types of blood cells or blood ingredient produce different optical rotation signals, the blood vessel signal analyzers can be set to receive and analyze the special optical rotation signal which is used as the blood vessel signal of the person A to be measured. The blood vessel signal analyzer can be Oxy-meter, Oxyhemoglobin, Deoxyhemoglobin, Carboxyhemoglobin, Methemoglobin analyzers, Carbon Dioxide Meter, or other detectors or analyzers for detecting or analyzing substances level or signal in blood. The first and second blood vessel signal analyzers 11, 12 are used to detect the wave signal of the blood vessels (including heat vessel and body vessel). The first and second blood vessel signal detectors 13 and 14 are located a predetermined distance from each other.

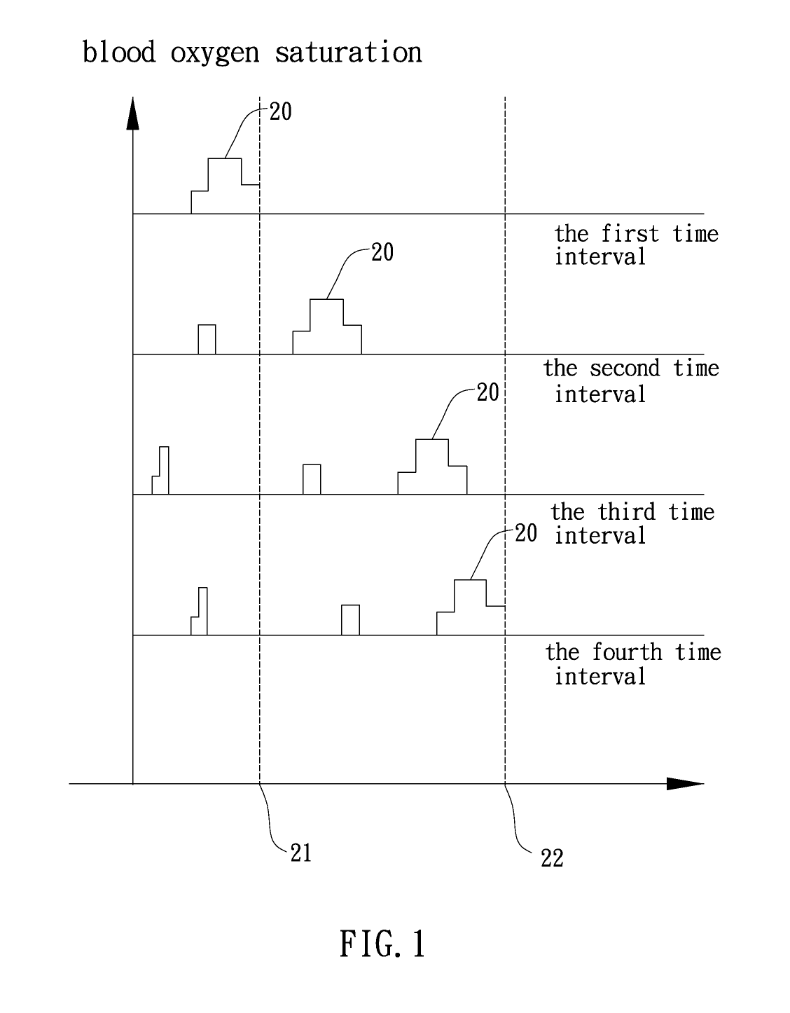

The first and second blood vessel signal analyzers 11 and 12 record the blood signal of the person A to be measured at a predetermined time interval, for example, the first and second blood vessel signal analyzers 11, 12 are oxy-meters and set to record the blood oxygen saturation of the person A to be measured. For a better understanding of technical characteristic of the present invention, please refer to FIG. 1, which is a graph showing the blood oxygen saturation values recorded during a first, second, third and fourth time intervals, respectively, when blood flow velocity is measured.

After that, when the special blood vessel signal (which refers to the blood oxygen saturation value in this embodiment and won't be furthered explained hereafter), for example, a recognizable blood oxygen saturation value signal 20 as shown in FIG. 1 appears in the record of the first and second blood vessel analyzers 11, 12, since the first blood vessel signal detector 13 is located upstream of the predetermined position along the blood flow direction (at the position 21 where the first blood vessel signal detector is worn or placed), and the second blood vessel signal detector 14 is located upstream along the blood flow direction (at the position 22 where the second blood vessel signal detector is worn or placed), the recognizable blood oxygen saturation value signal 20 (which might be caused by any action of the user or by the user's reaction to external stimuli) will be detected by the first and second blood vessel signal detectors 13 and 14 (it is also feasible when the positions 21 and 22 where the first and blood vessel signal detectors are placed are exchanged, only the direction changes). In this embodiment, the first blood vessel signal detector 13 detects the recognizable blood oxygen saturation value signal 20 during the first time interval, the second blood vessel signal detector 14 detects the recognizable blood oxygen saturation value signal 20 during the fourth time interval, and the interval of time between the first and fourth time interval (which is obtained by deducting the first time interval from the fourth time interval) is set to be a predetermined time period.

Finally, dividing the value of the aforementioned predetermined distance by the value of the predetermined time period can get the average blood flow velocity between the first and second blood vessel signal detectors 13, 14. Preferably, the position 21 where the first blood vessel signal detector is placed can be adjusted as close as possible to the position 22 where the second blood vessel signal detector is placed as long as there is no interference between the first and second blood vessel signal detectors 13, 14, so that the average blood flow velocity measured will be very close to the real value of the blood velocity at the predetermined position at which the blood flow velocity is to be measured.

On the basis of the above conception, a blood flow velocity measuring device 1 in accordance with the present invention, as shown in FIG. 2, comprises: a controller 16, a first blood vessel signal analyzer 11, a second blood vessel signal analyzer 12, a first blood vessel signal detector 13 and a second blood vessel signal detector 14. The first and second blood vessel signal analyzers 11 and 12 are electrically connected to the controller 16, respectively, while the first and second blood vessel detectors 13, 14 are electrically connected to the first and second blood vessel signal analyzers 11, 12, respectively. The first blood signal detected and analyzed by the first blood vessel signal detector 13 and the first blood vessel signal analyzer 11 and the second blood signal detected and analyzed by the second blood vessel signal detector 14 and the first blood vessel signal analyzer 12 are all received and recorded by the controller 16, and the controller 16 then will calculate the blood flow velocity of the person to be measured based on the blood signals received. The calculation method is the same as above, so no further explanation is necessary.

The first and second blood vessel signal detectors 13, 14 are connected by a connecting member 15 for the purpose of obtaining the length between the first and second blood vessel signal detectors 13, 14, for instance, the length therebetween is set to be a predetermined distance. The connecting member 15 can also be a length-adjustable link rod, as shown in FIG. 3, so that the length between the first and second blood vessel signal detectors 13, 14 is adjustable to meet different measuring demands. Or, the connecting member 15 can be a telescopic structure.

The first and second blood vessel signal detectors 13, 14 are not limited to invasive and non-invasive types as long as they can detect the blood vessel signal of the human body. For example, they can be clamp type or loop type blood vessel signal detectors.

Based on the abovementioned technology, the method for measuring the blood flow velocity in accordance with the present invention is such that the person to be measured wears two blood vessel signal detectors which are located a predetermined distance from each other and placed upstream and downstream of the predetermined position on the user's body where the blood flow velocity is to be measured, along the blood flow direction. Since the blood signal will be transmitted in the blood flow direction, in this way, a predetermined time period can be obtained, and the blood flow velocity at the predetermined position on the user's body can be figured out. This method can figure out the blood flow velocity at the predetermined position on the user's body very quickly without requiring the user to do any movement or to respond to external stimuli. Hence, it is cheap but accurate and has the potential for wide application.

While we have shown and described various embodiments in accordance with the present invention, it is clear to those skilled in the art that further embodiments may be made without departing from the scope of the present invention.

What is claimed is:

1. A method for measuring blood flow velocity, comprising the steps of:
placing a first and second blood vessel signal detectors on a body of a person to be measured, wherein the first blood vessel signal detector is electrically connected to a first blood vessel signal analyzer, the second blood vessel signal detector is electrically connected to a second blood vessel signal analyzer, and the first and second blood vessel signal detectors are located a predetermined distance from each other;
using the first and second blood vessel signal analyzers to record the blood signal of the person to be measured at a predetermined time interval;
setting an interval of time from the moment a specific blood vessel signal appears in a record of the first blood vessel signal analyzer to the moment the specific blood vessel signal appears in a record of the second blood vessel signal analyzer to be a predetermined time period; and
dividing a value of the predetermined distance by a value of the predetermined time period can obtain a blood flow velocity between the first and second blood vessel signal detectors.

2. The method for measuring blood flow velocity as claimed in claim 1, wherein the blood flow velocity obtained is an average blood flow velocity between the first and second blood vessel signal detectors.

3. The method for measuring blood flow velocity as claimed in claim 1, wherein the first and second blood vessel signal detectors are connected by a connecting member.

4. The method for measuring blood flow velocity as claimed in claim 2, wherein the connecting member is a length-adjustable link rod or telescopic rod.

5. The method for measuring blood flow velocity as claimed in claim 1, wherein the first and second blood vessel signal detectors are of invasive or non-invasive type.

6. The method for measuring blood flow velocity as claimed in claim 1, wherein the first and second blood vessel signal detectors are of clamp or loop type.

7. The method for measuring blood flow velocity as claimed in claim 1, wherein the first and second blood vessel signal analyzers are used to analyze signals produced by blood flow and selected from the group consisting of oxy-meter, oxyhemoglobin analyzer, deoxyhemoglobin analyzer, carboxyhemoglobin analyzer, methemoglobin analyzer, and carbon dioxide meter.

8. The method for measuring blood flow velocity as claimed in claim 1, wherein the first and second blood vessel signal analyzers are used to analyze wave signals of blood.

9. The method for measuring blood flow velocity as claimed in claim 1, wherein a light beam of a predetermined wavelength is projected into blood vessel of the person to be measured to enable a specific type of blood cells or blood ingredient to produce an optical rotation signal which is used as a blood vessel signal value of the person to be measured and received by the blood vessel signal analyzers.

10. A device for measuring blood flow velocity comprising:
a controller;
a first blood vessel signal analyzer electrically connected to the controller;
a second blood vessel signal analyzer electrically connected to the controller;
a first blood vessel signal detector electrically connected to the first blood vessel analyzer; and
a second blood vessel signal detector electrically connected to the second blood vessel analyzer;
wherein a first blood signal detected and analyzed by the first blood vessel signal detector and the first blood vessel signal analyzer and a second blood signal detected and analyzed by the second blood vessel signal detector and the first blood vessel signal analyzer are all recorded by the controller, and then the controller calculate a blood flow velocity of the person to be measured.

11. The method for measuring blood flow velocity as claimed in claim 10, wherein the blood flow velocity calculated by the controller is an average blood flow velocity between the first and second blood vessel signal detectors.

12. The device for measuring blood flow velocity as claimed in claim 9, wherein the first and second blood vessel signal detectors are connected by a connecting member.

13. The device for measuring blood flow velocity as claimed in claim 9, wherein the connecting member is a length-adjustable link rod or telescopic rod.

14. The device for measuring blood flow velocity as claimed in claim 9, wherein the first and second blood vessel signal detectors are of invasive or non-invasive type.

15. The device for measuring blood flow velocity as claimed in claim 9, wherein the first and second blood vessel signal detectors are of clamp or loop type.

16. The device for measuring blood flow velocity as claimed in claim 9, wherein the first and second blood vessel signal analyzers are used to analyze signals produced by blood flow and selected from the group consisting of oxy-meter, oxyhemoglobin analyzer, deoxyhemoglobin analyzer, carboxyhemoglobin analyzer, methemoglobin analyzer, and carbon dioxide meter.

17. The device for measuring blood flow velocity as claimed in claim 9, wherein the first and second blood vessel signal analyzers are used to analyze wave signals of blood.

18. The device for measuring blood flow velocity as claimed in claim 9, wherein a light beam of a predetermined wavelength is projected into blood vessel of the person to be measured to enable a specific type of blood cells or blood ingredient to produce an optical rotation signal which is used as a blood vessel signal value of the person to be measured and received by the blood vessel signal analyzers.
